(19) United States
(12) Patent Application Publication
(10) Pub. No.: US 2014/0051941 A1
(43) Pub. Date: Feb. 20, 2014

(54) OBTAINING PHYSIOLOGICAL MEASUREMENTS USING A PORTABLE DEVICE

(71) Applicant: Robert G. Messerschmidt, Los Altos, CA (US)

(72) Inventor: Robert G. Messerschmidt, Los Altos, CA (US)

(73) Assignee: Rare Light, Inc., Mountain View, CA (US)

(21) Appl. No.: 13/588,902

(22) Filed: Aug. 17, 2012

(51) Int. Cl. A61B 5/0205; A61B 5/1455; A61B 5/024; A61B 5/01; A61B 5/053; A61B 5/0404; A61B 5/103; A61B 5/00; A61B 5/04

(52) U.S. Cl. 600/301

(57) ABSTRACT

An apparatus and method for obtaining one or more physiological measurements associated with a user using a portable device alone or in combination with a detachable unit is disclosed herein. One or more of different types of sensor sets are included in one or more planar surfaces of the portable device and/or the detachable unit in communication with the portable device. The accuracy of physiological measurements is automatically ensured by the fixed positioning of the sensors relative to each other. A variety of different physiological measurements can be obtained using a portable device that users normally carry around and use on a daily basis, instead of requiring use of a separate/dedicated medical device.

### TECHNICAL FIELD

The present disclosure relates to obtaining physiological measurements in general, and in particular embodiments, to obtaining physiological measurements using a portable device.

### BACKGROUND

The current standard of care for blood pressure measurement is using a brachial cuff in the doctor's office or at home. Brachial cuff measurements comprise oscillometric measurements in which an air inflated cuff is positioned radially around a patient's arm in the vicinity of his/her brachial artery. Using a brachial cuff, however, is cumbersome and inadequate for a number of reasons. The cuff is uncomfortable and may even cause bruising. Brachial cuff measurements are susceptible to motion artifacts. Air pressure cuff devices tend to be large and not amendable to miniaturization. Brachial cuff measurements are also inadequate for thoroughly understanding a patient's blood pressure and changes in blood pressure. High blood pressure can be missed at the doctor's office if a patient's blood pressure is only high at certain times of the day. In this case, the opportunity to diagnose and treat high blood pressure is missed. Conversely, the patient may exhibit high blood pressure only when at the doctor's office. In this case, the patient may be unnecessarily placed on daily medication to lower blood pressure. Moreover, brachial cuff measurements provide peripheral blood pressure measurements (e.g., blood pressure at the arteries in the arms or legs) which can differ from central blood pressures (e.g., blood pressure at or near the aorta). For diagnostic and treatment purposes, central blood pressure measurements are preferred because they are a more accurate indicator of cardiovascular health.

Increasingly, the standard of care is moving toward ambulatory, non-invasive methods of obtaining physiological measurements. In the case of blood pressure measurements, a plurality of measurements obtained over a 24 hour or longer time period are of increasing importance in the practice of medicine. Such measurements provide better diagnosis and/or treatment of cardiovascular problems. Blood pressure is an important health statistic for overall health and wellness. When miniaturizing or configuring blood pressure measuring devices for home use, increasing their accuracy is an important consideration. Especially since patients are less well-versed in how to take measurements than medical personnel, it would be beneficial for measurement accuracy to be more or less built into the measurement device.

Other types of physiological measurements that may be tracked by individuals over an extended period of time and which are of value for overall health and wellness include, but are not limited to, electrocardiogram (ECG), body fat, and body water content measurements. So that individuals need not carry around multiple devices, it would be beneficial if a single device could capture one or more types of physiological measurements. It would also be beneficial if individuals can use an already existing device, which they would carry around anyway, to additionally perform physiological measurement functions.

### BRIEF SUMMARY

In certain embodiments, a portable device obtains one or more psychological measurements associated with a user. In some embodiments, the portable device is configured to be a handheld device. The portable device may be a unitary structure, or may include a base unit and a detachable unit. For example, the base unit may contain at least a portion of the processing capability and, in some embodiments a user interface such as a touch screen display; and the detachable unit might include sensors for the physiological measurements. For either configuration, the sensors have fixed positioning and distance on a rigid planar surface of the portable device (or detachable unit, as appropriate). Such sensor configuration automatically increases measurement accuracy, decreases improper sensor positioning, and the like. Moreover, the user's natural gripping motion of a handheld portable device provides automatic additional sensor contact locations to ensure contact with body parts on each of the left and/or right sides of the user's body. The processing and communication capabilities of the portable device can be harnessed to provide a beginning-to-end measurement experience to the user. Physiological measurements include, but are not limited to, blood pressure measurements, ECG measurements, heart rate measurements, body temperature measurements, galvanic skin response measurements, stress level indications, body water content measurements, and/or body fat content measurements.

Other features and aspects of the invention will become apparent from the following detailed description, taken in conjunction with the accompanying drawings which illustrate, by way of example, the features in accordance with embodiments.

### BRIEF DESCRIPTION OF THE DRAWINGS

Some embodiments are illustrated by way of example and not limitations in the figures of the accompanying drawings, in which:

FIGS. 1A-1B illustrates embodiments of an example system for obtaining one or more types of physiological measurements according to some embodiments.

FIGS. 2A-2D illustrates example portable devices of FIGS. 1A-1B used to obtain physiological measurements according to some embodiments.

FIG. 3 illustrates the portable device in contact with a body part of a user to obtain a physiological measurement (e.g., blood pressure) according to some embodiments.

FIG. 4 illustrates the portable device in contact with the user to obtain one or more physiological measurements (e.g., blood pressure, temperature, electrocardiogram (ECG), body fat content, body water content, heart beat, etc.) according to some embodiments.

FIGS. 5A-5C illustrates an example flow diagram for obtaining physiological measurements using the system of FIGS. 1A-1B according to some embodiments.

FIG. 6 illustrates an example block diagram showing modules configured to facilitate the process of flow diagram 500 according to some embodiments.

FIGS. 7A-7D illustrates user interface screens provided on the portable device 101 to provide physiological parameters capture instructions to the user according to some embodiments.

FIG. 8 illustrates blood pulse waveforms detected by a pair of optical sensors in accordance with some embodiments.

FIG. 9 depicts a block diagram representation of an example architecture for the controller assembly according to some embodiments.

The headings provided herein are for convenience only and do not necessarily affect the scope or meaning of the terms used.

### DETAILED DESCRIPTION

The following detailed description refers to the accompanying drawings that depict various details of examples selected to show how the present invention may be practiced. The discussion addresses various examples of the inventive subject matter at least partially in reference to these drawings, and describes the depicted embodiments in sufficient detail to enable those skilled in the art to practice the invention. Many other embodiments may be utilized for practicing the inventive subject matter than the illustrative examples discussed herein, and many structural and operational changes in addition to the alternatives specifically discussed herein may be made without departing from the scope of the inventive subject matter.

In this description, references to “one embodiment” or “an embodiment,” or to “one example” or “an example” mean that the feature being referred to is, or may be, included in at least one embodiment or example of the invention. Separate references to “an embodiment” or “one embodiment” or to “one example” or “an example” in this description are not intended to necessarily refer to the same embodiment or example; however, neither are such embodiments mutually exclusive, unless so stated or as will be readily apparent to those of ordinary skill in the art having the benefit of this disclosure. Thus, the present invention can include a variety of combinations and/or integrations of the embodiments and examples described herein, as well as further embodiments and examples as defined within the scope of all claims based on this disclosure, as well as all legal equivalents of such claims.

For the purposes of this specification, a “processor-based system” or “processing system” as used herein, includes a system using one or more microprocessors, microcontrollers and/or digital signal processors or other devices having the capability of running a “program,” (all such devices being referred to herein as a “processor”). A “program” is any set of executable machine code instructions, and as used herein, includes user-level applications as well as system-directed applications or daemons.

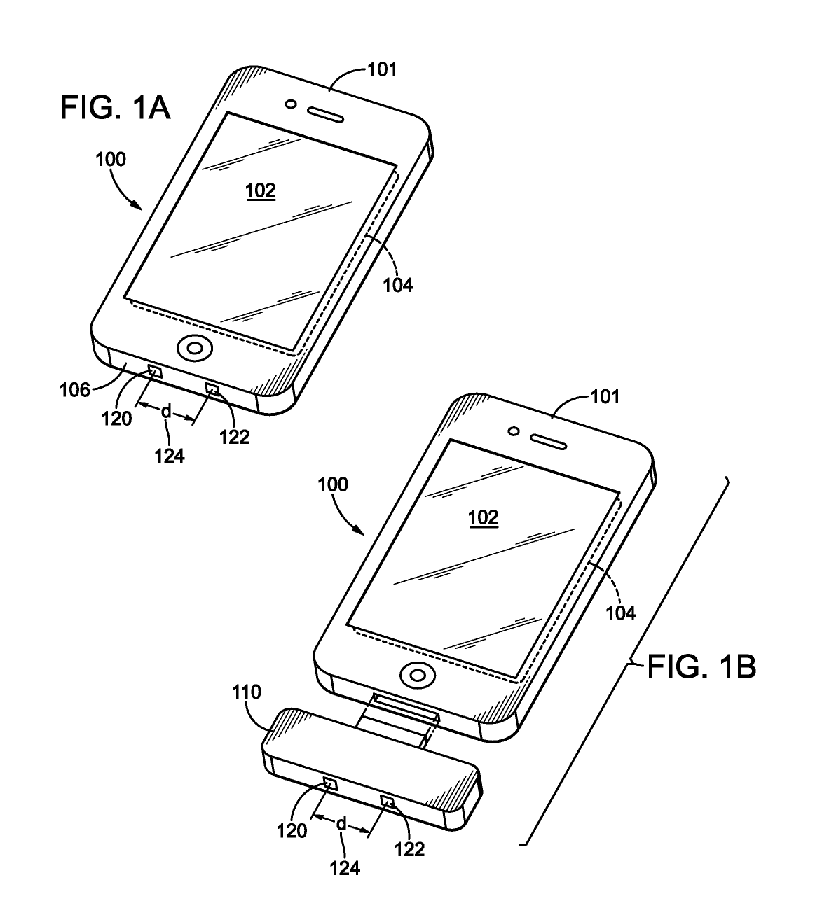

FIGS. 1A and 1B illustrate examples of a system 100 for obtaining one or more types of physiological measurements according to some embodiments. In FIG. 1A, one embodiment of the system 100 comprises a portable device 101. The portable device 101 of FIG. 1 includes a touch sensor panel 102 (also referred to as a touch screen) and a controller assembly 104. The touch sensor panel 102 includes an array of pixels to sense touch event(s) from a user's finger, or other body part, or a stylus or similar object. Examples of touch sensor panel 102 includes, but is not limited to, capacitive touch sensor panels, resistive touch sensor panels, infrared touch sensor panels, and the like. The controller assembly 104 is configured to provide processing and control capabilities for the portable device 101. The controller assembly 104 can include, but not limited to, machine-executable instructions, software applications (apps), circuitry, and the like.

The portable device 101 also includes a first sensor 120 spaced a fixed, known distance 124 apart from a second sensor 122, both sensors provided on a same planar surface of the portable device 101 (e.g., a bottom 106). The first and second sensors 120, 122 can be provided on any surface, such as the front, back, top, bottom, or any side edge, of the portable device 101. The plane of the portable device 101 containing both of the first and second sensor 120, 122 is placed in contact with a body part proximate to a major artery to optically obtain blood pressure measurements. Examples of suitable body parts include, but are not limited to, the upper arm (containing a brachial artery), wrist (containing radial and ulnar arteries), chest (containing an ascending aorta), neck (containing a carotid artery), or leg (containing a femoral artery).

FIG. 1B shows an alternative embodiment of the system 100 comprising the portable device 101 and a detachable unit 110. In this embodiment, the first and second sensors 120, 122 are located on a planar surface of the detachable unit 110 instead of the portable device 101. (The physiological measurement obtained from the first and second sensors 120, 122 provided on the detachable unit 110, nevertheless, is the same as when the sensors are provided on the portable device 101.) The first and second sensors 120, 122 can be provided on any surface of the detachable unit 110 that can be placed in contact with a body part containing a major artery, such as the front, back, top, bottom, or any side edge of the detachable unit 110. The detachable unit 110 can be detachably attached to one or more data ports of the portable device 101, for example, a 30-pin connector or universal serial bus (USB) port (either directly or via a cable therebetween). Alternatively, the detachable unit 110 can communicate with the portable device 101 using a wireless connection, such as Bluetooth. The detachable unit 110 can comprise, but is not limited to, a detachable dongle, cover/sleeve, or an accessory of the portable device 101.

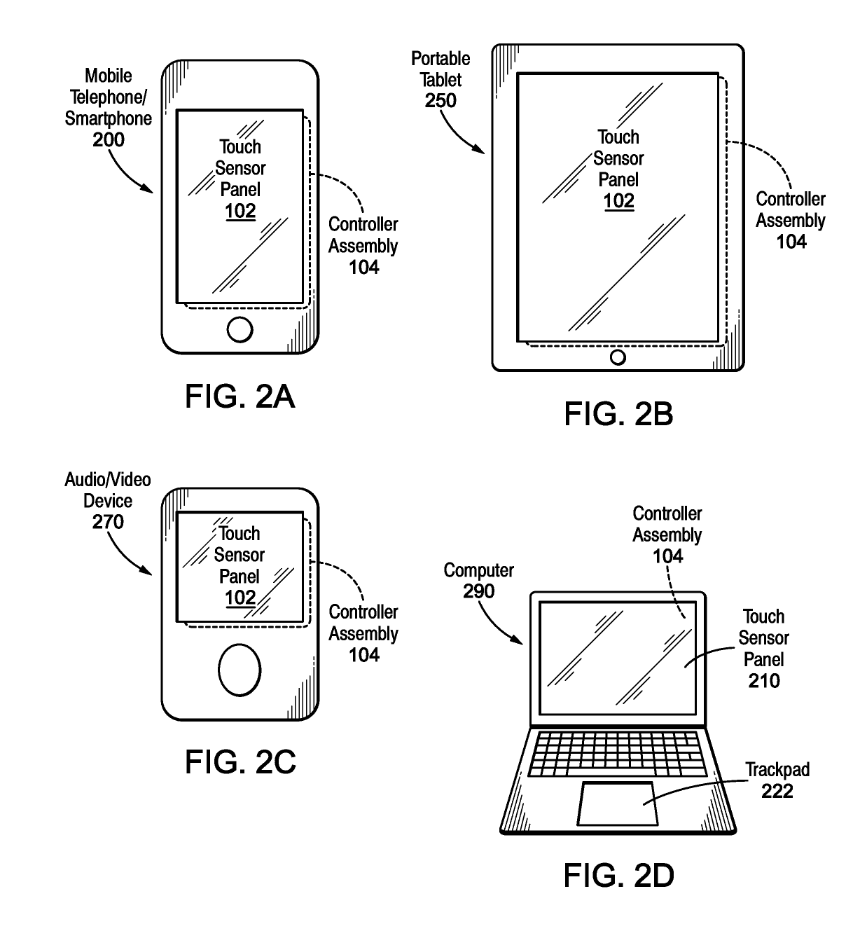

FIGS. 2A-2D illustrates examples of the portable device 101 according to some embodiments. A portable device includes any of a variety of processor-based devices that are easily portable to a user, including, for example, a mobile telephone or smart phone 200, a portable tablet 250, an audio/video device 270 (such as an iPod or similar multimedia playback device), a computer 290 such as a laptop or netbook, or a dedicated portable device specific for the purpose of making measurements of the types generally described herein (such as the detachable unit 110 in FIG. 1B); and further includes an external component that operatively couples to another portable device, such as through a USB port, a 30-pin port or another external interface port. Such external component can be in any of a variety of form factors, including a dongle coupled directly or through a cable to the port or another configuration that mechanically engages coupled portable device (such as a case structure, for example). Where one portable device is coupled to another portable device to function together, though each is a discrete “portable device,” the combination of the two devices should also be considered to be a “portable device” for purposes of this disclosure.

While many of the portable devices will be expected to include a touch screen, such is not necessarily required (see for example, computer 290 having a display 210, but not a touch screen), except for configurations herein which depend specifically on receiving inputs through such a touch screen, as will be apparent from the discussion to follow; though most embodiments will include some form of display though which to communicate with a user. Each of the portable devices includes a controller assembly 104 including one or more processors, which will provide the functionality of the device. Each portable device may also include additional controls or other components, such as: a power button, a menu button, a home button, a volume button, a camera, a light flash source for the camera, and/or other components to operate or interface with the device. In FIG. 2, the example touch screens 102 and controller assemblies 104 have been numbered similarly, though as will be readily apparent to those skilled in the art, such numbering is not intended to suggest that such structures will be identical to one another, but merely that the identified elements generally correspond to one another.

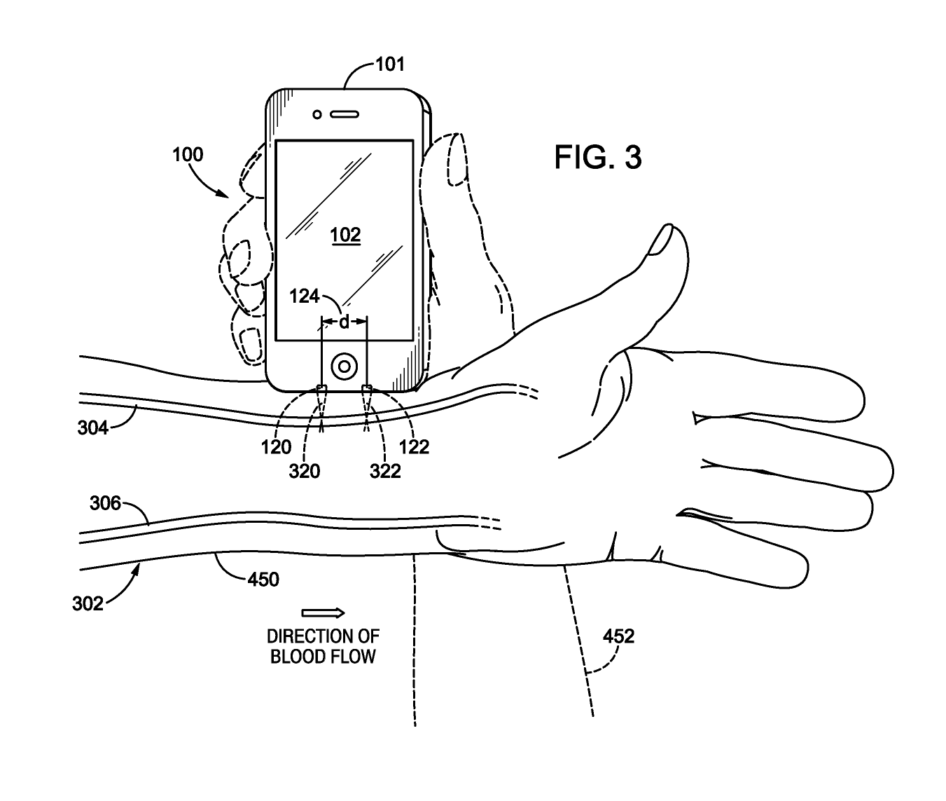

FIG. 3 illustrates the portable device 101 in contact with a body part of a user 302 to obtain a physiological measurement (e.g., blood pressure) relating to the user 302 according to some embodiments. The sensor set is shown exaggerated in FIG. 3A for ease of illustration. The bottom 106 plane of the portable device 101 is pressed against (e.g., is in pressure contact with) a wrist (or near the wrist or lower arm near the wrist) of the user 302. In this particular example, the skin of the wrist (or near the wrist) that is near the user's 302 thumb—as opposed to the inner wrist or the side of the wrist closest to the pinky finger—is contacted by the portable device 101 in order to measure the blood flow at a radial artery 304. The other artery located at the wrist is an ulnar artery 306. It is understood that a variety of body parts of the user 302 can similarly be contacted to obtain the physiological measurement.

Each of the first and second sensors 120, 122 comprises an optical type of sensor, and in particular, a reflective type photoplethysmography (PPG) sensor. Each of the first and second sensors 120, 122 includes a light source (e.g., a light emitting diode (LED)) and a photo detector. In each of the first and second sensors 120, 122, the light source and the photo detector are positioned relative to each other such that the portion of the light emitted by the light source that is reflected back by the body part can be captured by the photo detector.

In one embodiment, the wavelength of the light source of the first sensor 120 is different from the wavelength of the light source of the second sensor 120. For example, one of the first and second sensors 120, 122 can operate at about 630 nanometers (nm) and the other sensor can operate at about 820 nm. In another embodiment, both of the first and second sensors 120, 122 can operate at the same wavelength, such as about 940 nm. In either case, the wavelength(s) are selected to be within a range of approximately 600 to 900 nm. Skin is (sufficiently) transparent to and blood (sufficiently) absorbs light that is in the range of approximately 600 to 900 nm.

The remaining light beam characteristics are the same for the first and second sensors 120, 122. Each of a first light beam 320 for the first sensor 120 and a second light beam 322 for the second sensor 122 is configured to impinge the blood flowing in the radial artery 304 with minimal or no interference from each other. Each of the first and second light beams 320, 322 comprises a collimated or converging beam (with focal point within the radial artery 304). One or more lenses, collimator, or other optics can be provided at the output of the light source to achieve a desired beam width and/or minimize one beam crossing over into the detection area of the other sensor. The power requirement of each of the first and second sensors 120, 122 is low, in the order of a few milliWatts (mW).

The distance 124 is a fixed, known distance selected based on a number of factors. The distance 124 is configured to be small enough so that when the side of the portable device 101 with the first and second sensors 120, 122 contacts the skin, both sensors likely experience the same or nearly the same degree of contact pressure and coupling with the skin and the radial artery 304. Generally the smaller the distance, the better the possibility of achieving similar contact pressure and coupling for both sensors. The distance 124 is also configured to be not too small so as to cause overlap between the first and second light beams 320, 322. The beam width of each of the first and second light beams 320, 322 is configured to be a small percentage of the distance 124, such as 5%. Generally the greater the distance 124 relative to the beam width, less care can be taken regarding the beam profiles of the first and second light beams 320, 322. As an example, the distance 124 can be 10-25 mm.

By fixing the locations of the first and second sensors 120, 122 relative to each other (and by extension the distance 124 therebetween), the uncertainty of the distance traveled by the blood pulse between the two sensors common in traditional pulse oximetry is automatically eliminated. Knowing the exact distance aids in accuracy of the blood pressure measurement. Moreover, having a relatively small distance also facilitates similar contact pressure between the sensor and the skin for both sensors also facilitates accuracy of the blood pressure measurement.

Accordingly, as discussed in detail below, each of the first and second sensors 120, 122 is configured to measure the blood pulses arriving at the respective portions of the radial artery 304 as a function of time. A given blood pulse arrives first at the portion of the radial artery 304 irradiated by the first sensor 120 (because this portion of the radial artery 304 is closer to the user's 302 heart), and then travels to the portion of the radial artery 304 irradiated by the second sensor 122. In other words, there is a time delay between the given blood pulse arriving at each of the first and second sensors 120, 122. This time delay or difference is referred to as a difference in a pulse arrival time (Δ PAT) or a difference in a pulse transit time (Δ PTT). The Δ PAT is then converted into a blood pressure measurement.

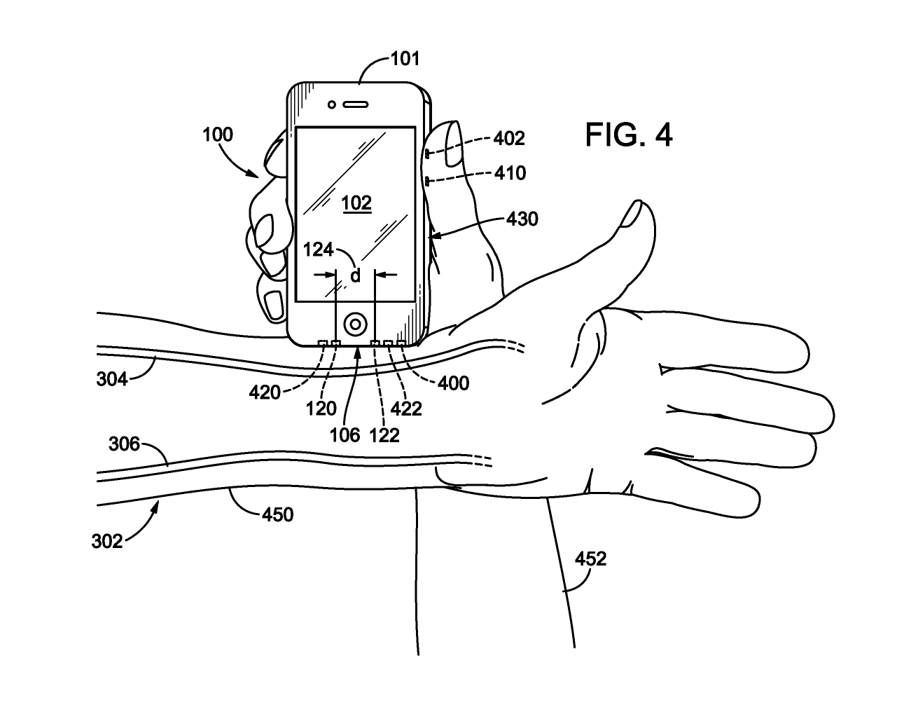

FIG. 4 illustrates the portable device 101 in contact with the user 302 to obtain one or more physiological measurements (e.g., blood pressure, temperature, electrocardiogram (ECG), body fat content, body water content, heart beat, etc.) according to some embodiments. In FIG. 4, the first and second sensors 120, 122 (separated by the distance 124), a first electrode 400, a third electrode 420, and a fourth electrode 422 are provided on a same planar surface of the portable device 101 (e.g., bottom 106). A second electrode 402 and a temperature sensor 410 are provided on another same planar surface of the portable device 101 (e.g., a side edge 430).

Each of the first and second sensors 120, 122; first, second, third, and fourth electrodes 400, 402, 420, 422; and temperature sensor 410 can be located on any surface, such as the front, back, top, bottom, or any side edge, of the portable device 101. All of the first and second sensors 120, 122; first, second, third, and fourth electrodes 400, 402, 420, 422; and temperature sensor 410 can be located on the same surface of the portable device 101 relative to each other, except that the first and second electrodes 400, 402 are located relative to each other so as to respectively contact opposite sides of the user's 302 body (e.g., left and right sides of the user's 302 body such as the left and right extremities) and the third and fourth electrodes 420, 422 are positioned (on the same planar surface of the portable device 101) to both contact the same side of the user's 302 body. The temperature sensor 410 is provided, for example, on the side edge 430 with the second electrode 402 because of space constraints on the bottom 106. The location of and/or the distance between each of the sensors/electrodes relative to each other on a given planar surface (with the exception of the first and second sensors 120, 122) is not limited to that shown in FIG. 4.

The bottom 106 of the portable device 101 is placed in contact with the skin of the wrist (or near the wrist or lower arm near the wrist) proximate to the radial artery 304 (similar to the contact in FIG. 3). All of the first and second sensors 120, 122 and first, third, and fourth electrodes 400, 420, 422 provided on the bottom 106 are thus in contact with the user's 302 skin and proximate to the radial artery 304 of a left arm 450 of the user 302. The portable device 101 is held against the wrist area by a right hand 452 of the user 302. The natural holding/gripping motion of the portable device 101 causes portions of the right hand 452 to make contact with the second electrode 402 and temperature sensor 410 located on the side 420. Note that one contact area of the user's 302 body (e.g., left arm 452) is across the torso of the other contact area of the user's 302 body (e.g., right hand 452), the relevance of which is explained below.

The first, second, third, and fourth electrodes 400, 402, 420, 422 (also referred to as sensors, conductors, conductive electrodes, contact locations, contact regions, contact areas, etc.) comprises a conductive material such as, but not limited to, a metallic material, conductive hydrogel, silicon, conductive yarns including silver coated nylon, stainless steel yarn, silver coated copper filaments, silver/silver chloride, and the like. The temperature sensor 410 can comprise a thermocouple, thermopile, or resistance temperature detector (RTD) type of sensor. The first and second electrodes 400, 402 are configured to obtain ECG, heart rate, body water content, and/or body fat content measurements. The temperature sensor 410 is configured to obtain a (skin surface) temperature measurement, a type of body temperature measurement. The third and fourth electrodes 420, 422 are configured to obtain a galvanic skin response measurement.

Although the system 100 of FIG. 4 comprises the portable device 101 including various types of sensors/electrodes, it is understood that one or more of these sensors/electrodes can be located on the detachable unit 110 and one or both of the portable device 101 and the detachable unit 110 may be used to obtain the physiological parameters corresponding to the physiological measurements. Moreover, less than four sets of sensors/electrodes may be included in the portable device 101 and/or detachable unit 110, in any combination with each other.

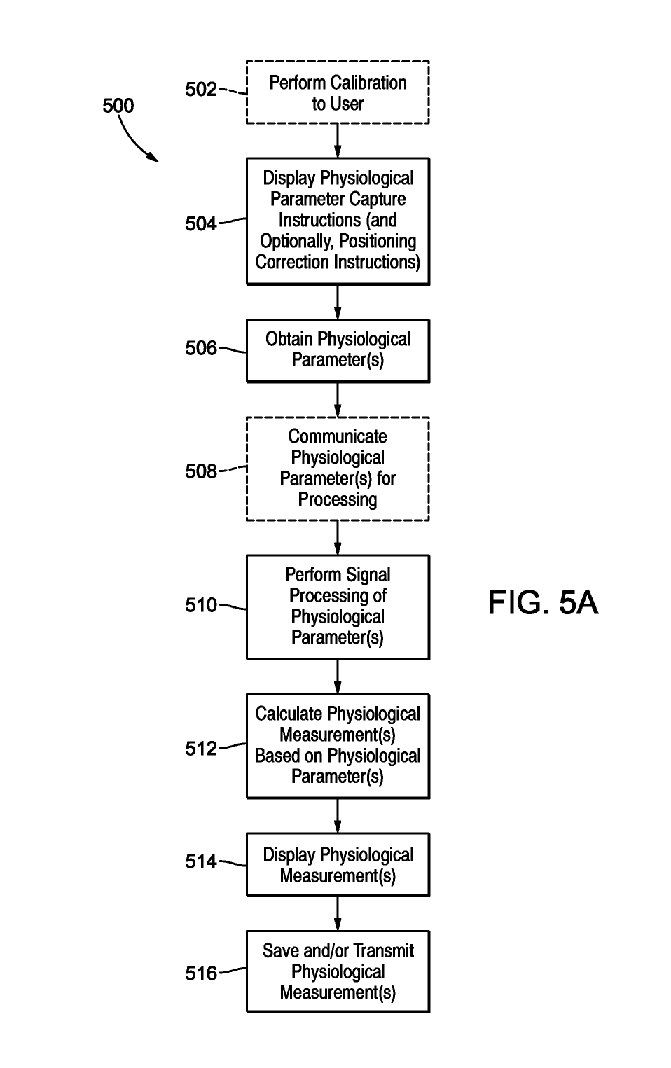

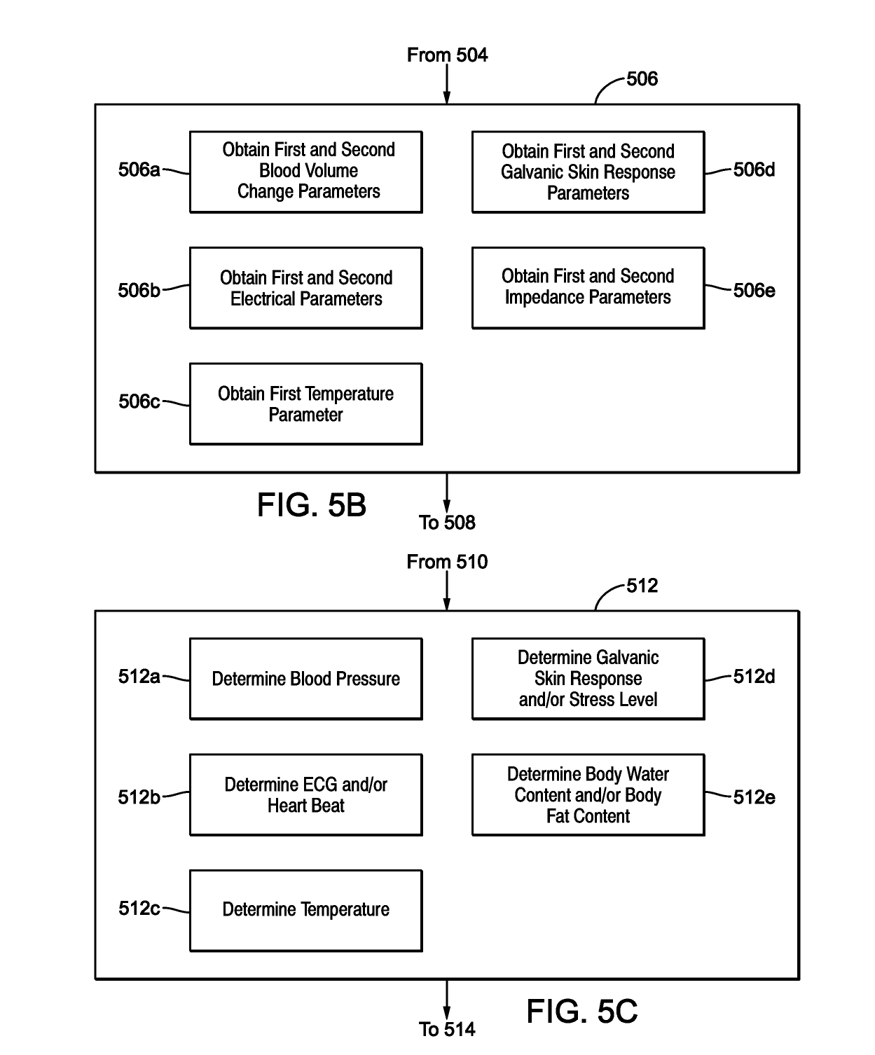

FIGS. 5A-5C illustrates an example flow diagram 500 for obtaining physiological measurements using the system 100 according to some embodiments. FIG. 6 illustrates an example block diagram showing modules configured to facilitate the process of flow diagram 500 according to some embodiments. The modules shown in FIG. 6 are included in the controller assembly 104 of the portable device 101. The modules of FIG. 6 comprise conceptual modules representing instructions encoded in a computer readable storage device. When the information encoded in the computer readable storage device are executed by the controller assembly 104, computer system or processor, it causes one or more processors, computers, computing devices, or machines to perform certain tasks as described herein. Both the computer readable storage device and the processing hardware/firmware to execute the encoded instructions stored in the storage device are components of the portable device 101. Although the modules shown in FIG. 6 are shown as distinct modules, it should be understood that they may be implemented as fewer or more modules than illustrated. It should also be understood that any of the modules may communicate with one or more components external to the portable device 101 via a wired or wireless connection, such as the detachable unit 110. FIGS. 5A-5C will be described in conjunction with FIG. 6.

At a block 502, a calibration module 602 is configured to perform calibration with respect to the user 302 in preparation of obtaining usable physiological measurement(s). The need to perform calibration depends on the type of physiological measurement to be obtained. In one embodiment, calibration is performed for measurements that use blood pulse transit time or blood pulse velocity that is converted into central aortic blood pressure measurements. An information display module 604 may be configured to cause the portable device 101 to display calibration instructions on the touch sensor panel 102. For example, the calibration instructions may instruct the user 302 to use a brachial cuff to obtain one or more blood pressure measurements while simultaneously having the first and second sensors 120, 122 obtain physiological parameters (e.g., blood pulse waveforms as a function of time). The brachial cuff blood pressure measurement(s) may be automatically transmitted to the portable device 101, or the portable device 101 may provide input fields on the touch sensor panel 102 for the user 302 to manually input the blood pressure obtained from the brachial cuff.

At or approximately the same time that the brachial cuff measurement(s) is being made, the portable device 101 (or the detachable unit 110, as appropriate) is configured to obtain one or more blood pressure measurements using the first and second sensors 120, 122. Using both sets of blood pressure measurements, the calibration module 502 is configured to determine one or more scaling factor to properly calibrate the conversion of the blood pulse transit time (or blood pulse velocity) obtained using the first and second sensors 120, 122 from the user 302 to a central (e.g., aortic) blood pressure measurement. The conversion function between the blood pulse transit time (or blood pulse velocity) and desired blood pressure measurement is known, as discussed in detail below, but the scaling up or down of the conversion function for each particular user is obtained from the calibration process.

In another embodiment, calibration is performed for physiological measurements using skin impedance detection (e.g., body fat content measurement). The information display module 604 may be configured to cause display of calibration instructions relating to skin impedance measurements on the touch sensor panel 102. Calibration instructions may instruct the user 302 to enter his/her height, weight, age, and gender prior to measuring the user's 302 skin impedance. The calibration module 602 is configured to use the user-specific information to calibrate the user's skin impedance measurement to report an accurate body fat content information to the user 302.

The type of calibration(s) may be automatically determined based on the types of sensor(s) provided on the portable device 101 and/or detachable unit 110. Alternatively, the calibration(s) are performed based on the types of physiological measurements specified by the user 302. One or more calibration may be performed at the block 502 for a particular user. Calibration may be performed each time before a physiological measurement is made, it may be performed periodically (e.g., once a month), or it may be a one-time event for a given user. The calibration schedule for one type of physiological measurement may be the same or different from another type of physiological measurement.

In still another embodiment, the calibration block 302 may be omitted. For example, in the case of electrocardiogram (ECG) measurements, no calibration with respect to particular individuals is required to calculate an ECG measurement from electro-physiological parameters detected from individuals. As another example, no calibration may be required for providing body temperature measurements to users. As still another example, if it is assumed that peripheral blood pressure (e.g., radial blood pressure) is the same or sufficiently the same as central aortic blood pressure or peripheral blood pressure is the desired physiological measurement, then calibration for determining blood pressure may be omitted.

Next at a block 504, the information display module 604 is configured to cause display of physiological parameter(s) capture instructions on the touch sensor panel 102. The physiological parameters capture instructions comprise one or more user interface screens providing instructions, tips, selection options, and other information to the user 302 to facilitate proper detection of physiological parameter(s) corresponding to desired physiological measurement(s).

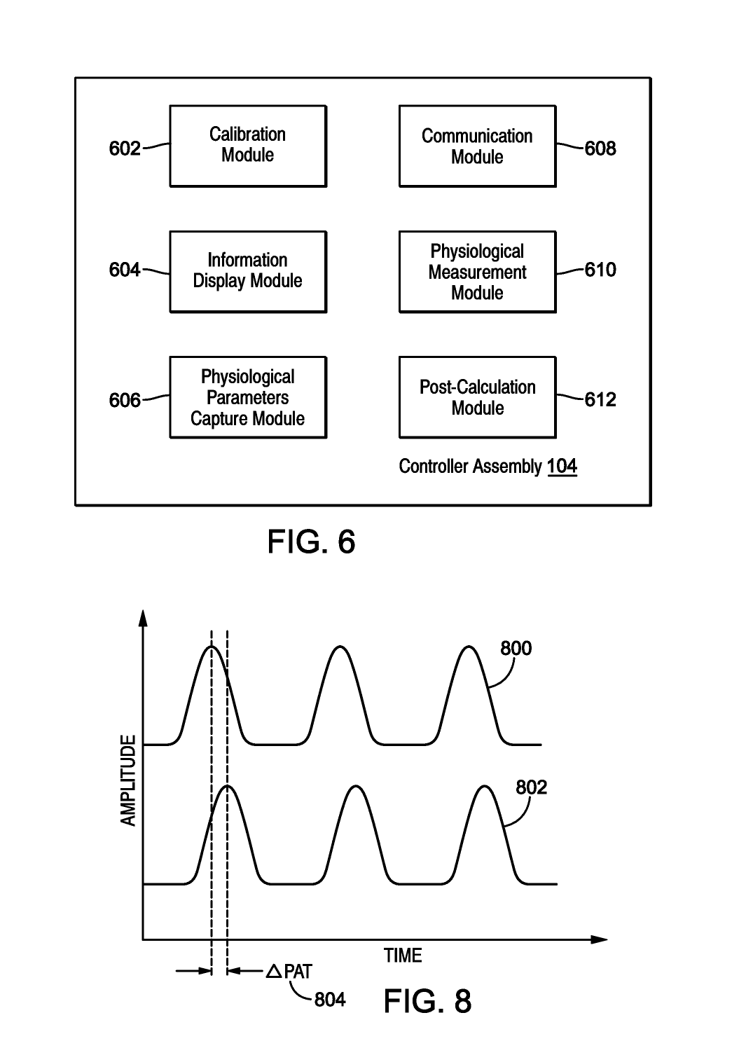

In one embodiment, a user interface screen 702 (FIG. 7A) at the portable device 101 provides measurement selection options to the user 302. The user 302 can select one or more physiological measurements such as, but not limited to, blood pressure, ECG, heart beat, body temperature, galvanic skin response/stress level, body water content, body fat content, etc. Next at a user interface screen 704 (FIG. 7B), instructions on how to hold and place the portable device 101 with respect to the user 302 is provided. A user interface screen 706 (FIG. 7C) provides additional instructions to achieve proper positioning and contact between the sensors/electrodes included in the portable device 101 and the user 302. The user interface screen 706 may be provided in response to an indication that one or more of the sensors/electrodes (corresponding to those measurements selected by the user 302 in the user interface screen 702) is not detecting physiological parameters or the detected signals are incorrect (out of range, too low signal, etc.). As an example, if contact with the first and/or second sensors 120, 122 is improper, a user interface screen 708 can be provided to the user 302 to interactively aid in proper positioning of the first and second sensors 120, 122 to a particular portion of the user's 302 body to obtain an accurate blood pressure measurement.

The amount of skin-to-sensor contact pressure with which each of the first and second sensors 120, 122 contacts the user 302 is proportional to the amplitude of the respective blood pulse waveforms detected by the first and second sensors 120, 122. The greater the contact pressure for a given sensor, the greater the amplitude of that sensor's detected blood pulse waveform. The distance 124 between the first and second sensors 120, 122 is selected to be small enough such that both sensors are likely to experience similar contact pressures when the bottom 106 containing both sensors is placed in contact with the user 302. However, in the case that sufficiently different contact pressure is detected between the two sensors (via differences in their respective blood pulse waveform amplitudes), then a real-time graphic (e.g., a pair of bars) indicative of the amount of contact pressure for each of the first and second sensors 120, 122 can be provided to aid the user 302 to correct positioning of the portable device 101. The real-time graphic can also be used to guide the user 302 to find the desired peripheral artery. For example, if the user 302 initially places the portable device 101 against a portion of the left lower arm that is not proximate to the radial artery 304 or the ulnar artery 306, then the first and second sensors 120, 122 would detect no blood pulses and the real-time graphic can correspondingly register such low or no signal state. The portable device 101 can guide the user 302 to move the portable device 101 until appropriate blood pulses are detected.

In another embodiment, the user interface screen 702 can be omitted since the portable device 101 is configured to automatically provide the physiological measurements based on whatever sets of sensors/electrodes are provided on the portable device 101. In still another embodiment, the portable device 101 can be configured to perform a check on the adequacy of the signals detected by the appropriate sensors/electrodes included in the portable device 101, but only provide the user interface screen 708 (or other similar user interface screens) if inadequate signals are detected.

Next at a block 506, a physiological parameters capture module 606 is configured to control the sensors/electrodes provided on the portable device 101 corresponding to the physiological measurements designated (implicitly or explicitly) in the block 504, to cause those sensors/electrodes to obtain physiological parameter(s) from the user 302. The physiological parameters capture module 606 provides the necessary input, timing, and/or power signals to these sensors/electrodes for periodic or continuous data capture.

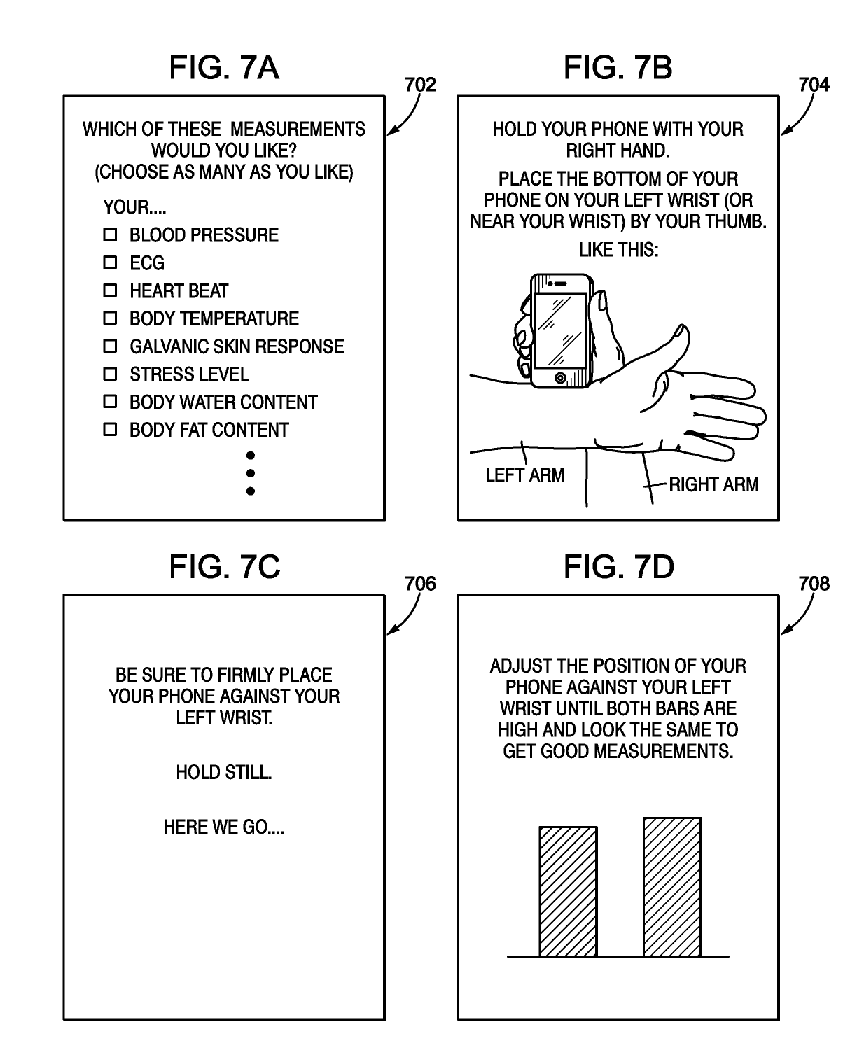

FIG. 5B illustrates example sub-blocks 506a-e of the block 506 according to some embodiments. At a sub-block 506a, the physiological parameters capture module 606 is configured to obtain a first blood volume change parameter from the first sensor 120 and a second blood volume change parameter from the second sensor 122. When the first light beam 320 emitted from the first sensor 120 enters the user's 302 body, it is transmitted through the skin (and other structures between the surface of the user's 302 body to the radial artery 304) to be absorbed by the blood arriving at a first particular portion of the radial artery 304. Some of the first light beam 320, however, is not absorbed but is instead reflected by one or more physiological structures below the surface of the skin back toward the first sensor 120. The reflected portion of the first light beam 320 is detected by the photo detector included in the first sensor 120. The changing blood volume at the first particular portion of the radial artery 304 as a function of time is caused by the blood pulses arriving at that particular portion of the radial artery 304 as a function of time. The change in the blood volume as a function of time causes the reflected portion of the first light beam 320 to correspondingly change over time, the resulting reflected light resembling a train of light pulses. The first sensor 120 thus detects changes in the reflected light over time corresponding to a first blood pulse waveform 800, as shown in FIG. 8. The amplitude or magnitude of the first blood pulse waveform 800 is proportional to the contact pressure between the first sensor 120 and the user's 302 body.

A second blood pulse waveform 802 is similarly obtained from the second sensor 122 based on the reflected portion of the second light beam 322 at a second particular portion of the radial artery 304, the peaks of the second blood pulse waveform 802 shifted in time (by an amount Δ PAT 804) relative to the peaks of the first blood pulse waveform 800. This time difference between the two waveforms exists because a given blood pulse arrives first at the first particular portion of the radial artery 304 corresponding to the first sensor 120 before it arrives at the second particular portion of the radial artery 304 corresponding to the second sensor 122.

At a sub-block 506b, the physiological parameters capture module 606 is configured to simultaneously obtain a first electrical parameter from the first electrode 400 and a second electrical parameter from the second electrode 402. An electrical circuit is completed by the first electrode 400, the second electrode 402, and the user 302. The first electrode 400 makes electrical contact with a portion of the user's left arm 450 while the second electrode 402 makes electrical contact with a portion of the user's right arm (e.g., right hand 452), as shown in FIG. 4. The first and second electrodes 400, 402 obtain resistive measurements from one side of the user's body to the other side, which are converted into ECG and/or heart beat measurements.

At a sub-block 506c, the physiological parameters capture module 606 is configured to obtain a first temperature parameter from the temperature sensor 410. The first temperature parameter comprises a skin surface temperature associated with the user 302. Skin (surface) temperature relates, among other things, to the user's stress level. Typically in a stressful situation, a person's peripheral circulation (including skin circulation) decreases, which causes the skin temperature to decrease.

At a sub-block 506d, the physiological parameters capture module 606 is configured to obtain both a first galvanic skin response parameter from the third electrode 420 and a second galvanic skin response parameter from the fourth electrode 422. An electrical circuit is completed by the third electrode 420, the fourth electrode 422, and the user 302. Both of the third and fourth electrodes 420, 422 are configured to make electrical contact with the user's left arm 450 (e.g., on the same side of the user's body), as shown in FIG. 4. The third and fourth electrodes 420, 422 obtain (skin) impedance measurements corresponding to the moisture level of the user's skin at the contact areas, the moisture level indicative of a galvanic skin response. Galvanic skin response, in turn, is an indication of a person's stress level (or the opposite of stress, relaxation level).

At a sub-block 506e, the physiological parameters capture module 606 is configured to obtain a first impedance parameter from the first electrode 400 and a second impedance parameter from the second electrode 402. The first and second electrodes 400, 402 operate on the circuit-completion concept to obtain impedance measurements between one side of the user's body to the other side. Such measurements are converted into body water content measurements and/or body fat content measurements.

Returning to FIG. 5A, once one or more of the physiological parameter(s) have been obtained, if such parameters were captured from sensors/electrodes located on the detachable unit 110, these parameters are communicated from the detachable unit 110 to the portable device 101 (block 508). The physiological parameters can be provided to the portable device 101 via a wire connection (e.g., data ports such as the 30-pin connector or USB port) or wireless connection (e.g., Bluetooth). Depending on the frequency of the physiological parameters from a given set of sensors/electrodes and/or the number of types of physiological parameters from different set of sensors/electrodes, physiological parameters from a given set of sensors/electrodes can be singularly provided to portable device 101 (e.g., in real- or near real-time) or it can be combined with physiological parameters from one or more of other sets of sensors/electrodes for a combined transmission to the portable device 101. A communication module 608 is configured to coordinate communication of obtained physiological parameters from the detachable unit 110 to the portable device 101.

Next at a block 510, a physiological measurement module 610 is configured to control signal processing and other pre-processing functions to ready the obtained physiological parameters suitable for conversion to appropriate physiological measurements. Depending on the state of the physiological parameters received at the portable device 101, one or more of the following processing functions may occur: analog-to-digital (A/D) conversion, demultiplexing, amplification, one or more filtering (each filter configured to remove a particular type of undesirable signal component such as noise), other pre-conversion processing, and the like. The processing can be performed by hardware, firmware, and/or software. The type and extent of signal processing can vary depending on the type of physiological parameters. For example, physiological parameters obtained from the first and second sensors 120, 122 may undergo digitization, filtering, and other signal conditioning. Whereas physiological parameters obtained from the first and second electrodes 400, 402 may require little signal processing, e.g., merely A/D conversion. Additionally, in some embodiments, some or all of the signal processing may be performed by the sensors/electrodes themselves. For example, if the raw output of a certain sensor requires signal processing unique to that sensor (e.g., unique circuitry) and/or the sensor packaging can easily include signal processing functionalities, the raw output of a sensor may be processed by the sensor itself. An advantage of this approach is that the portable device 101 requires less circuitry, for example, that is dedicated for one function especially if the sensor set is located at the detachable unit 110. Another advantage is that the portable device 101 may receive uniform physiological parameters from a variety of sensor sets.

Next at a block 512, the physiological measurement module 610 is configured to determine appropriate physiological measurements from the (conditioned) physiological parameters. Block 512 comprises additional processing to translate physiological parameters into physiological measurements that are well-understood by the user 302. FIG. 5C illustrates example sub-blocks 512a-e of the block 512 according to some embodiments. Like suffix in sub-blocks 512a-e and sub-blocks 506a-e correspond with each other (e.g., sub-block 512a corresponds to sub-block 506a). Each of the sub-blocks 512a-e comprise use of a particular algorithmic method or functional relationship(s) established between given physiological parameters and physiological measurements to convert or translate those physiological parameters to appropriate physiological measurements.

At the sub-block 512a, the physiological measurement module 610 is configured to determine a central (aortic) blood pressure measurement based on the first and second blood volume change parameters obtained from the first and second sensors 120, 122. The first and second blood volume change parameters comprise the first and second blood pulse waveforms 800, 802, respectively (see FIG. 8). As shown in FIG. 8, Δ PAT 804 is derived from the first and second blood pulse waveforms 800, 802. The distance between the first and second sensors 120, 122 is known—distance 124. Thus, a pulse wave velocity (PWV) is the difference in the distance between the first and second sensors 120, 122 divided by the difference in the pulse transit time between the first and second sensors 120, 122: PWV=distance 124/Δ PAT 804. The PWV relates to the central aortic blood pressure (also referred to as the central arterial blood pressure (CABP)): PWV=f(CABP).

In one embodiment, the translation or conversion of PWV to CABP can be performed using known algorithmic methods that specify the quantitative relationship or correlation between PWV and CABP. As an example, reference is made to http://en.wikipedia.org/wiki/Pulse_wave_velocity that provides example algorithmic methods for the functional relationship between PWV and CABP. The article includes the following equation showing the relationship between PWV and P (arterial blood pressure CABP):

$$PWV = \sqrt{\frac{d P \cdot V}{ρ \cdot d V}} ,$$

where P is the density of blood and V is the blood volume. The article also provides an alternative expression of PWV as a function of P (arterial blood pressure CABP):

PWV=Pᵢ/(νᵢ·ρ)=Z_{c}/ρ,

where ν is the blood flow velocity (in the absence of wave reflection) and ρ is the density of blood.

In another embodiment, the functional relationship between Δ PAT (or PWV) and CABP can be empirically derived. For example, a human study can be conducted in which three simultaneous measurements are obtained from each subject: (1) Δ PAT via the first and second sensors 120, 122, (2) a CABP by actually measuring the blood pressure at the subject's aorta during cardiac catheterization (adding a pressure sensor to a catheter that is snaked through the subject's arteries, including positioning the pressure sensor on the catheter in the subject's aortic arch to directly measure CABP), and (3) a brachial blood pressure (brachial BP) using a brachial cuff. A relatively small number of subjects are sufficient, such as about 50 subjects. The three simultaneous measurements for a given subject provide an empirical relationship between Δ PAT, CABP, and brachial BP. The empirical relationships from all the subjects are averaged, resulting in an empirically-derived functional relationship between Δ PAT and CABP. Alternatively, two simultaneous measurements (Δ PAT via the first and second sensors 120, 122, and CAPB using cardiac catheterization) are sufficient to determine the correlation between Δ PAT and CABP.

The empirically-derived relationship between Δ PAT, CABP, and brachial BP can also be used to calibrate each particular user from which Δ PAT will be obtained. In particular, as discussed above with respect to block 502, a Δ PAT measurement and a brachial BP measurement are simultaneously obtained from a given user during calibration. Using these two known measurements associated with the given user in comparison with the derived functional relationship between Δ PAT and brachial BP, a scaling factor applicable to the particular user can be determined. The scaling factor typically adjusts the CABP up or down in value. Subsequently, when a Δ PAT measurement is actually obtained from that user using the first and second sensors 120, 122, the portable device 101 can convert the measured Δ PAT to a provisional brachial BP using the derived functional relationship between Δ PAT and brachial BP and additionally apply the (calibration) scaling factor applicable to that user to the provisional brachial BP to determine a final brachial BP. The final brachial BP, in turn, is converted into the CABP using the derived functional relationship between brachial BP and CABP.

In still another embodiment, the physiological measurement module 610 is configured to determine a peripheral blood pressure measurement using the calculated PWV. When the first and second sensors 120, 122 contact the left arm 450 proximate the radial artery 304, the physiological measurement module 610 is configured to determine a radial blood pressure measurement. It may be assumed that the peripheral blood pressure and central blood pressure are sufficiently the same for a given user so that conversion to a central blood pressure is unnecessary.

At the sub-block 512b, the physiological measurement module 610 is configured to determine an ECG and/or heart beat measurement based on the first electrical parameter from the first electrode 400 and the second electrical parameter from the second electrode 402. In one embodiment, the ECG measurements comprise Lead 1 ECG signal measurements. The detected Lead 1 ECG signals may undergo little or no processing/conversion to form the final ECG measurements. In another embodiment, the Lead 1 ECG signals may be converted into a heart rate measurement (also referred to as a pulse measurement) using known algorithmic methods. An example algorithmic method is discussed at http://en.wikipedia.org/wiki/Electrocardiography. An example algorithmic method is discussed at http://courses.kcumb.edu/physio/ecg%20primer/normecgcalcs.htm#The %20R-R %20interval/, which discusses identifying a particular point on consecutive signals of the ECG waveform and using the known time difference between such particular points on the consecutive signals to obtain the number of heart beats per unit of time.

At the sub-block 512c, the physiological measurement module 610 is configured to determine a skin surface temperature measurement or stress/relaxation level indication based on the first temperature parameter obtained from the temperature sensor 410. In one embodiment, the first temperature parameter undergoes little or no processing/conversion to output a skin surface temperature measurement. As an example, the skin temperature may merely be a conversion of the first temperature parameter in accordance with a conversion table or equation. In another embodiment, a known or empirically-derived correlation between the skin surface temperature and stress level can be used to provide a stress/relaxation level indication based on the first temperature parameter (or a series of temperature readings). An example discussion of the relationship is provided in Lawrence Baker et al., “The relationship under stress between changes in skin temperature, electrical skin resistance, and pulse rate,” Journal of Experimental Psychology, Vol. 48(5), 361-366 (November 1954). The Baker article discusses a study in which subjects were subjected to stress stimuli and corresponding quantitative changes to skin temperature from a rest/baseline state were recorded. The study revealed that there was significant increase in skin temperature under stress stimulation.

At the sub-block 512d, the physiological measurement module 610 is configured to determine a galvanic skin response measurement or stress/relaxation level indication based on the first and second galvanic skin response parameters obtained from the third and fourth electrodes 420, 422. The first and second galvanic skin response parameters comprise a measure of the moisture level of the user's skin at the contact areas, and galvanic skin response is indicative of stress/relaxation level. Known or empirically-derived correlations between the skin moisture level, galvanic skin response, and stress/relaxation levels can be used to translate the first and second galvanic skin response parameters into the galvanic skin response measurement and/or stress/relaxation level indication. An example discussion of the relationship is provided in: Marjorie K. Toomin et al., “GSR biofeedback in psychotherapy: Some clinical observations,” Psychotherapy: Theory, Research & Practice, Vol. 12(1), 33-38 (Spring 1975). The Toomin article describes a study in which the galvanic skin response of subjects was manipulated using attention, excitation, or emotional provoking stimuli. The study observed that that the amount of reaction (change in galvanic skin response relative to a baseline) to a given stimuli across different subjects was variable—subjects could be classifies as over-reactors, under-reactors, or variable-reactors. This suggests that a series of galvanic skin response measurements may be made to determine a baseline for the user before indications of stress/relaxation levels start being provided to the user. For instance, assuming that stress stimuli in the real world are infrequent events, if a user has frequent significant changes in galvanic skin response, this may indicate that the user is a variable-reactor or over-reactor such that measurements of high (or non-insignificant) change after the baseline measurement period may not necessarily indicate stress. Conversely, a user who shows little change over time (e.g., an under-reactor) that registers a high (or non-insignificant) change after the baseline measurement period may actually be indicative of stress.

At the sub-block 512e, the physiological measurement module 610 is configured to determine a body fat content measurement and/or a body water content measurement based on the first and second impedance parameters obtained from the first and second electrodes 400, 402. Use of body impedance information to generate physiological measurement comprises bioelectrical impedance analysis (BIA) measurements. For at least the body fat content measurement, the first and second impedance parameters may be converted to corresponding body fat content using known algorithmic methods, such algorithmic method taking into account the user's weight, height, gender, and/or age (previously provided by the user 302 at calibration block 502). In other embodiments, known algorithmic methods may be used for each of body fat content and body water content determination without calibration information. Examples of suitable algorithmic methods for body fat content determination are provided in Ursula G. Kyle et al., “Bioelectrical impedance analysis—part I: review of principles and methods,” Clinical Nutrition, Vol. 23 (5): 1226-1243 (2004), and G. Bedogni et al., “Accuracy of an eight-point tactile-electrode impedance method in the assessment of total body water,” European Journal of Clinical Nutrition, Vol. 56, 1143-1148 (2002) (available at http://www.nature.com/ejcn/journal/v56/n11/full/1601466a.html) for body water content determination. Tables 2 and 3 of the Kyle article provide a survey of equations reported in other articles for calculating the body fat as a function of the subject's measured resistance (which is quantitatively related to impedance), height, weight, age, gender, and/or other variables. Since these equations provide an estimation of the body fat, the amount of error inherent in each of the equations is also provided in the tables. For body water content determination, the Bedogni article provides tables and plots to empirically translate measured resistance for a certain body part (e.g., trunk, right arm, left arm, right leg, left leg) to a resistance value for the whole body and from that to the body water content value (referred to as total body water (TBW) in the article).

With the determination of physiological measurement(s) completed in block 512, the information display module 604 is configured to facilitate display of one or more user interface screens including such physiological measurement(s) on the touch sensor panel 102 (block 514). Associated information about the presented physiological measurement(s) may also be provided on the touch sensor panel 102 to aid the user 302 in understanding the measurements. For blood pressure measurements, for example, different range values and what each range means may be provided and for those range values indicative of health issues, recommendations may be given to see a doctor right away or the like.

Last, at a block 516, the calculated physiological measurement(s) along with related information (e.g., time and date stamp, user identifier, etc.) can be saved in the portable device 101 and/or transmitted to another device. A post-calculation module 612 is configured to facilitate saving the data to a memory included in the portable device 101. The post-calculation module 612 is also configured to facilitate transmission of the physiological measurement(s) (and their associated information) over a network, such as over a cellular network or a WiFi network, to a remote device (e.g., another portable device, server, database, etc.). By saving and/or communicating one or more physiological measurements over time, such information may illuminate trends for useful health assessment.

It is understood that one or more of blocks 502-516 may be performed in a different sequence than shown in FIG. 5A. For example, block 516 may be performed prior to or simultaneously with block 514. Sub-blocks 512a-e of FIG. 5C may be performed in any sequential order or simultaneously with each other depending on, for example, when a set of physiological parameters are received by the portable device 101 and/or the processing capacity of the portable device 101.

In this manner, a portable device alone or in combination with a detachable unit obtains one or more psychological measurements associated with a user. Unlike with traditional measurement methods, the fixed positioning and distance inherently provided by situating sensors on a rigid planar surface of the portable device (or detachable unit, as appropriate) automatically increases measurement accuracy, decreases improper sensor positioning, and the like. Moreover, the user's natural gripping motion of the portable device provides automatic additional sensor contact locations to ensure contact with body parts on each of the left and right sides of the user's body. The processing and communication capabilities of the portable device can be harnessed to provide a beginning-to-end measurement experience to the user. Physiological measurements include, but are not limited to, blood pressure measurements, ECG measurements, heart rate measurements, body temperature measurements, galvanic skin response measurements, stress level indications, body water content measurements, and/or body fat content measurements.

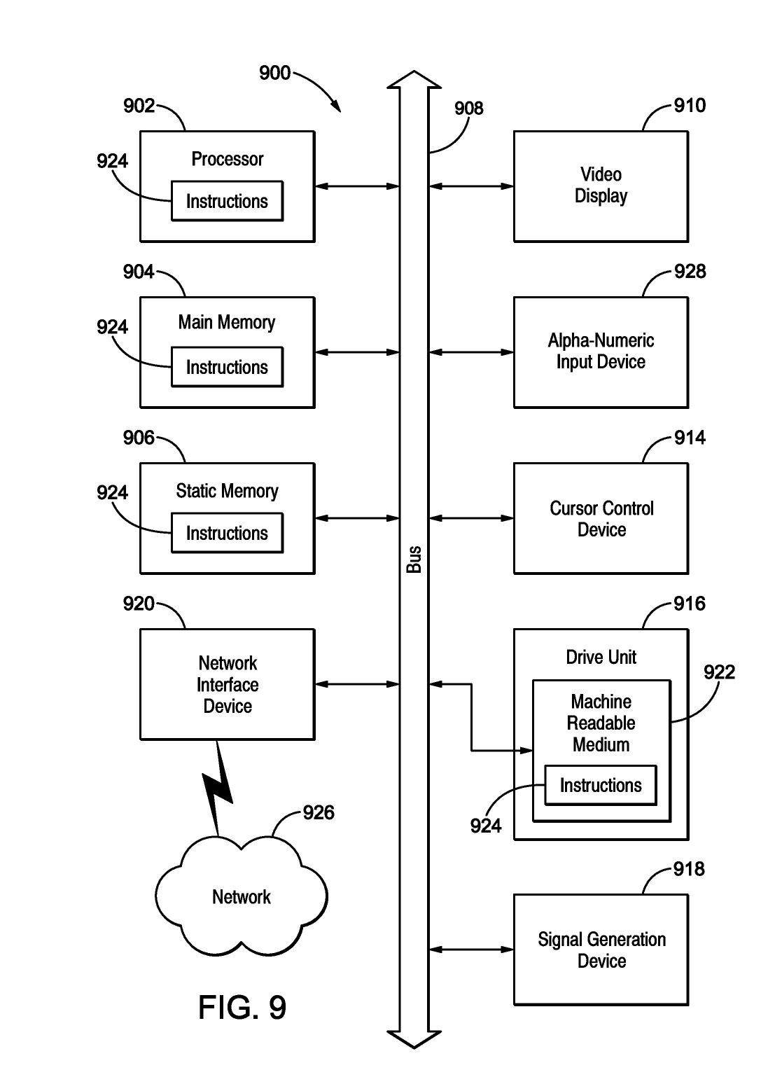

FIG. 9 depicts a block diagram representation of an example architecture for the controller assembly 104. Although not required, many configurations for the controller assembly 104 can include one or more microprocessors which will operate pursuant to one or more sets of instructions for causing the machine to perform any one or more of the methodologies discussed herein.

An example controller assembly 900 includes a processor 902 (e.g., a central processing unit (CPU), a graphics processing unit (GPU) or both), a main memory 904 and a static memory 906, which communicate with each other via a bus 908. The controller assembly 900 may further include a video display unit 910 (e.g., a liquid crystal display (LCD) or a cathode ray tube (CRT)). The controller assembly 900 may also include an alphanumeric input device 912 (e.g., a keyboard, mechanical or virtual), a cursor control device 914 (e.g., a mouse or track pad), a disk drive unit 916, a signal generation device 918 (e.g., a speaker), and a network interface device 920.

The disk drive unit 916 includes a machine-readable medium 922 on which is stored one or more sets of executable instructions (e.g., apps) embodying any one or more of the methodologies or functions described herein. In place of the disk drive unit, a solid-state storage device, such as those comprising flash memory may be utilized. The executable instructions may also reside, completely or at least partially, within the main memory 904 and/or within the processor 902 during execution thereof by the controller assembly 900, the main memory 904 and the processor 902 also constituting machine-readable media. Alternatively, the instructions may be only temporarily stored on a machine-readable medium within controller 900, and until such time may be received over a network 926 via the network interface device 920.

While the machine-readable medium 922 is shown in an example embodiment to be a single medium, the term “machine-readable medium” as used herein should be taken to include a single medium or multiple media (e.g., a centralized or distributed database, and/or associated caches and servers) that store the one or more sets of instructions. The term “machine-readable medium” or “computer-readable medium” shall be taken to include any tangible non-transitory medium (which is intended to include all forms of memory, volatile and non-volatile) which is capable of storing or encoding a sequence of instructions for execution by the machine.

Many additional modifications and variations may be made in the techniques and structures described and illustrated herein without departing from the spirit and the scope of the present invention. Accordingly, the present invention should be clearly understood to be limited only by the scope of the claims and equivalents thereof.

What is claimed is:

1. A hand-held device for obtaining one or more physiological measurements, comprising:
a touch-sensitive display;
a first sensor on a first surface of the device;
a second sensor on the first surface of the device, the first sensor and the second sensor separated by a fixed distance on the first surface; and
a processor in communication with the touch-sensitive display, the first sensor, and the second sensor, wherein the processor is configured to process a first physiological parameter associated with the first sensor and a second physiological parameter associated with the second sensor to determine a physiological measurement.

2. The hand-held device of claim 1, wherein the first surface comprises a continuous surface of the handheld device.

3. The hand-held device of claim 2, wherein the handheld device comprises a detachable unit in communication with a base unit including the processor and the touch-sensitive display.

4. The hand-held device of claim 2, wherein the detachable unit of the handheld device comprises the first surface.

5. The hand-held device of claim 1, wherein each of the first and the second sensors comprises a reflective type photoplethysmography (PPG) sensor and the physiological measurement comprises a blood pressure measurement.

6. The hand-held device of claim 1, wherein the first and the second physiological parameters comprise blood pulse parameters associated with a peripheral artery of a user.

7. The hand-held device of claim 1, wherein the touch-sensitive display is configured to display instructions for a user to provide the first and the second physiological parameters.

8. The system of claim 1, further comprising:
a first electrode provided on the first surface, the first electrode configured to obtain a third physiological parameter; and
a second electrode provided on a second surface, the second electrode configured to obtain a fourth physiological parameter, and wherein the processor is configured to determine a second physiological measurement based on the third and the fourth physiological parameters.

9. The system of claim 8, wherein the first surface and the second surface comprise a same surface of the device.

10. The system of claim 9, wherein the second physiological measurement comprises one of a galvanic skin response measurement and a stress level indication.

11. The system of claim 8, wherein the first surface and the second surface comprise different surfaces of a device, and wherein the third physiological parameter and the fourth physiological parameters each comprise parameters from respective portions of a user's body located at opposite sides of the user's torso.

12. The system of claim 11, wherein the second physiological measurement comprises one of an electrocardiogram (ECG) measurement, a heart rate measurement, a body water content measurement, and a body fat content measurement.

13. The system of claim 1, further comprising a temperature sensor in communication with the processor, the processor configured to determine a body temperature measurement based on a temperature parameter associated with a user provided by the temperature sensor.

14. The system of claim 1, further comprising a transmitter in communication with the processor, the transmitter configured to transmit the physiological measurement to a remote device.

15. A handheld portable device, comprising:
a touch-sensitive display;
a first sensor located at a first surface of the portable device;
a second sensor located at the first surface of the portable device, the first sensor and the second sensor separated by a fixed distance at the first surface; and
a processor in communication with the touch-sensitive display, wherein the processor is configured to determine a first physiological measurement based on a first physiological parameter from the first sensor and a second physiological parameter from the second sensor.

16. The handheld portable device of claim 15, wherein the portable device comprises a smart phone, a tablet, a laptop, a portable music device, a portable video device, or a computing device.

17. The handheld portable device of claim 15, wherein each of the first and the second sensors comprises a reflective type photoplethysmography (PPG) sensor and the first physiological measurement comprises a blood pressure measurement.

18. The handheld portable device of claim 15, wherein the first and the second physiological parameters comprise blood pulse parameters associated with a peripheral artery of a user.

19. The handheld portable device of claim 15, wherein the touch-sensitive display is configured to display instructions for a user to provide the first and the second physiological parameters.

20. The handheld portable device of claim 15, further comprising:
a first electrode provided on the first surface and the first electrode configured to obtain a third physiological parameter; and
a second electrode provided on a second surface and the second electrode configured to obtain a fourth physiological parameter, wherein the processor is configured to determine a second physiological measurement based on at least one of the third and the fourth physiological parameters.

21. The handheld portable device of claim 20, wherein the first surface and the second surface comprise a same surface of the portable device, and wherein the second physiological measurement comprises one of a galvanic skin response measurement and a stress level indication.

22. The handheld portable device of claim 20, wherein the first surface and the second surface comprise different surfaces of a device, the third physiological parameter and the fourth physiological parameter comprising parameters from respective portions of a user's body located at opposite sides of the user's torso.

23. The handheld portable device of claim 20, wherein the first electrode comprises a temperature sensor and the second physiological measurement comprises a body temperature measurement.

24. A method for obtaining one or more physiological measurements, the method comprising:
displaying information on a touch-sensitive display of a portable device, the display information including identification of at least one portion of a user's body to be placed into contact with at least a first sensor and a second sensor provided on a same side of the device, the first sensor and the second sensor separated from each other by a fixed distance;
receiving, in response to the portion of the user's body in contact with the first sensor and the second sensor, a first physiological parameter associated with the first sensor and a second physiological parameter associated with the second sensor; and
generating a first physiological measurement based on the first physiological parameter and the second physiological parameter.

25. The method of claim 24, wherein each of the first and the second sensors comprises a reflective type photoplethysmography (PPG) sensor, and wherein the first physiological measurement comprises a blood pressure measurement.

26. The method of claim 24, further comprising:
receiving a third physiological parameter associated with a first electrode provided on the device;
receiving a fourth physiological parameter associated with a second electrode provided on the device; and
generating a second physiological measurement based on at least one of the third physiological parameter and the fourth physiological parameter.

27. The method of claim 26, wherein the second physiological measurement comprises an electrocardiogram (ECG) measurement, a heart beat measurement, a body temperature measurement, a galvanic skin response measurement, a stress level indication, a body water content measurement, or a body fat content measurement.
